(19) United States
(12) Patent Application Publication
(10) Pub. No.: US 2010/0286478 A1
(43) Pub. Date: Nov. 11, 2010

(54) FLEXIBLE SURGERY ACCESS SYSTEMS

(75) Inventors: Richard C. EWERS, Fullerton, CA (US); Barton P. BANDY, Orange, CA (US); Haio FAUSER, Encinitas, CA (US); Tung Thanh LE, Irvine, CA (US)

Correspondence Address: LEVINE BAGADE HAN LLP, 2400 GENG ROAD, SUITE 120, PALO ALTO, CA, 94303, US

(73) Assignee: USGI Medical, Inc., San Clemente, CA (US)

(21) Appl. No.: 12/766,731

(22) Filed: Apr. 23, 2010

Related U.S. Application Data
(60) Provisional application No. 61/172,170, filed on Apr. 23, 2009.

(51) Int. Cl. A61B 1/01

(52) U.S. Cl. 600/114

(57) ABSTRACT

A flexible surgery access and instrument management system includes a base unit and an insertion unit. The base unit provides a platform having a connection mechanism to which the insertion unit is attached. The insertion unit includes an elongated conduit having one or more tubes providing instrument passages, and a connection mechanism adapted to selectively couple with the mating connection mechanism provided on the base unit. The elongated conduit of the insertion unit is preferably steerable. One or more flexible instruments may be inserted through the tubes of the elongated conduit, with the proximal ends of the instruments being attached to the base unit such that the user is able to control and manipulate the instruments.

### RELATED APPLICATION DATA

This application claims the benefit of priority to U.S. Provisional Patent Application Ser. No. 61/172,170, filed on Apr. 23, 2009, the contents of which are incorporated herein by reference in its entirety.

### BACKGROUND

The present disclosure relates to systems and methods that provide surgical access to areas within the body of a patient through an endolumenal approach and/or through a limited number of access ports created by incisions in the patient's skin and/or internally communicating organ surfaces (e.g., stomach, rectum, vagina, etc.). More particularly, the present disclosure relates to systems and methods that provide surgical access and management of instruments used to perform diagnostic and therapeutic procedures in a minimally invasive manner.

Surgical techniques have evolved from open surgical procedures to less invasive procedures, including laparoscopy, endoscopy, and others. More recently developed techniques—including single port access (e.g., Single Incision Laparoscopic Surgery (SILS); One Port Umbilical Surgery (OPUS); Single Port Incisionless Conventional Equipment-utilizing Surgery (SPICES); or Natural Orifice TransUmbilical Surgery (NOTUS)) and natural orifice (e.g., Natural Orifice Translumenal Endoscopic Surgery (NOTES) or Natural Orifice Surgery (NOS)) techniques—reduce invasiveness further by reducing the number of access port sites down to just a single 2 cm or less skin incision or making small access incisions in internally communicating surfaces like the stomach, rectum, and/or vagina.

In view of these developments, there exists a need for new and improved surgical access devices, instruments, and instrument management systems that support minimally invasive surgical procedures.

### SUMMARY

In one general aspect, systems are provided for obtaining minimally invasive access to locations within the body of a patient using flexible diagnostic and/or therapeutic instruments and tools. The systems include surgical access devices that are able to be advanced to a surgical location within the body of a patient through a single point natural orifice and/or incision location and to provide a conduit for surgical necessities such as light and visualization devices, insufflation devices, suction and irrigation, surgical tools, and other instruments. In some embodiments, the access devices are capable of being positioned (e.g., steerable) and of providing position stability.

Some embodiments of the system are resposable (reusable-in-part/disposable-in-part), having a reusable base unit and a disposable insertion unit. In those embodiments, the base unit is cleanable and sterilizable, and provides a stable platform having a connection mechanism to which the insertion unit may be attached. The insertion unit includes an elongated conduit having one or more tubes providing instrument passages, and a connection mechanism adapted to selectively couple with the mating connection mechanism provided on the base unit. The system thereby provides access for flexible diagnostic and therapeutic instruments in a minimally invasive manner.

In several embodiments, the base unit is suitable for use as a user interface that allows the system to be used either in an endoscopic “hand held” condition or in a surgical “stand held” condition. In both conditions, the system provides the user with the capability to push, pull, and/or twist the insertion portion of the device for access and positioning in the human body. The system also provides the user with the ability to manage, park, govern, and/or secure any surgical instrument(s) that are used in association with the system.

In another general aspect, flexible instrument management systems and methods of their use are provided that provide the ability of the user to correct or modify the images viewed through an endoscope such that the images are more intuitively associated with the movement and operation of the instruments being used with the system. The system includes a base unit and an insertion unit, with the insertion unit having a plurality of flexible tubes providing passages for an endoscope(s) and other flexible surgical (diagnostic and/or therapeutic) instruments. The system provides the ability for the user to adjust the positions of the endoscope and instruments to achieve the desired results.

In some embodiments, the system has the capability of pre-aligning a plurality of instrument tubes relative to an endoscope tube/endoscope, such as by installing the insertion unit into the base unit at a desired orientation. In other embodiments, the system has the capability of changing the location of one or more of the tube(s) carrying one or more of the instruments, thereby changing the user's point of interface with the instrument. In still other embodiments, an endoscope is able to be rotated or otherwise re-oriented within the system, thereby changing the orientation of the image displayed to the user by the endoscope. In still other embodiments, various combinations of the foregoing capabilities and methods are employed.

### BRIEF DESCRIPTION OF THE DRAWINGS

FIG. 1 is a perspective view of a flexible surgery access and instrument management system.

FIGS. 2A and 2B are a right-side and left-side perspective views of a base unit of the flexible surgery access and instrument management system of FIG. 1.

FIGS. 3A-C are rear views of a split collar clamp of the base unit of FIGS. 2A-B in the open, closed, and clamped positions, respectively.

FIGS. 4A-B are perspective views of a rotating clamp shown in an open and closed condition.

FIG. 5 is a perspective view of an insertion unit of the flexible surgery access and instrument management system of FIG. 1.

FIG. 6 is an end view of the elongated conduit of the insertion unit of FIG. 5.

FIG. 7A is a side perspective view of an insertion unit being brought into the vicinity of a base unit.

FIG. 7B is a side perspective view of the insertion unit and base unit of FIG. 7A being connected together.

FIG. 8 is a perspective view of a flexible surgery access and instrument management system including an endoscope.

FIGS. 9A and 9B are perspective views of a telescoping tube assembly.

FIGS. 10A and 10B are perspective views of a flexible instrument and an adapter for engaging the flexible instrument to a flexible surgery access and instrument management system.

FIGS. 11A and 11B are perspective views of alternative adapter embodiments.

FIGS. 12A and 12B are perspective views of another flexible instrument and another adapter for engaging the flexible instrument to a flexible surgery access and instrument management system.

FIGS. 13A and 13B are perspective views of an alternative embodiment of a flexible surgery access and instrument management system.

### DETAILED DESCRIPTION

Systems and methods for performing minimally invasive surgical procedures are described herein. The systems include surgical access devices that are able to be advanced to a surgical location within the body of a patient through a single point natural orifice and/or incision location and to provide a conduit for surgical necessities such as light and visualization devices, insufflation devices, suction and irrigation, surgical tools, and other instruments. In some embodiments, the access devices are capable of being positioned (e.g., steerable) and of providing position stability.

USGI Medical,Inc. of San Clemente, Calif. has developed several devices and methods that facilitate endoscopic, endolumenal, translumenal, and other minimally invasive diagnostic and therapeutic procedures. Several endoscopic access devices are described, for example, in the following United States patent applications:

TABLE 1

| US Pat. Appl. Ser. No. | Filing Date |
|---|---|
| 10/346,709 | Jan. 15, 2003 |
| 10/458,060 | Jun. 9, 2003 |
| 10/797,485 | Mar. 9, 2004 |
| 11/129,513 | May 13, 2005 |
| 11/365,088 | Feb. 28, 2006 |
| 11/738,297 | Apr. 20, 2007 |
| 11/750,986 | May 18, 2007 |
| 12/061,591 | Apr. 2, 2008 |

Several tissue manipulation and tissue anchor delivery devices are described in the following United States patent applications:

TABLE 2

| US Pat. Appl. Ser. No. | Filing Date |
|---|---|
| 10/612,109 | Jul. 1, 2003 |
| 10/639,162 | Aug. 11, 2003 |
| 10/672,375 | Sep. 26, 2003 |
| 10/734,547 | Dec. 12, 2003 |
| 10/734,562 | Dec. 12, 2003 |
| 10/735,030 | Dec. 12, 2003 |
| 10/840,950 | May 7, 2004 |
| 10/955,245 | Sep. 29, 2004 |
| 11/070,863 | Mar. 1, 2005 |
| 12/486,578 | Jun. 17, 2009 |

Endolumenal tissue grasping devices are described in several of the United States patent applications listed above, and in the following United States patent applications:

TABLE 3

| US Pat. Appl. Ser. No. | Filing Date |
|---|---|
| 11/736,539 | Apr. 17, 2007 |
| 11/736,541 | Apr. 17, 2007 |

Tissue anchors are described in several of the United States patent applications listed above, and in the following United States patent applications:

TABLE 4

| US Pat. Appl. Ser. No. | Filing Date |
|---|---|
| 10/841,411 | May 7, 2004 |
| 11/404,423 | Apr. 14, 2006 |
| 11/773,933 | Jul. 5, 2007 |

Additional endoscopic instruments are described in several of the United States patent applications listed above, and in the following United States patent applications:

TABLE 5

| US Pat. Appl. Ser. No. | Filing Date |
|---|---|
| 61/110,178 | Oct. 31, 2008 |

Endoscopic access systems, endosurgical instruments, and instrument management systems are described in several of the United States patent applications listed above, and in the following United States patent applications:

TABLE 6

| US Pat. Appl. Ser. No. | Filing Date |
|---|---|
| 12/138,348 | Jun. 12, 2008 |
| 61/116,955 | Nov. 21, 2008 |

Each of the foregoing patent applications is hereby incorporated by reference in its entirety.

### Flexible Surgery Access and Instrument Management

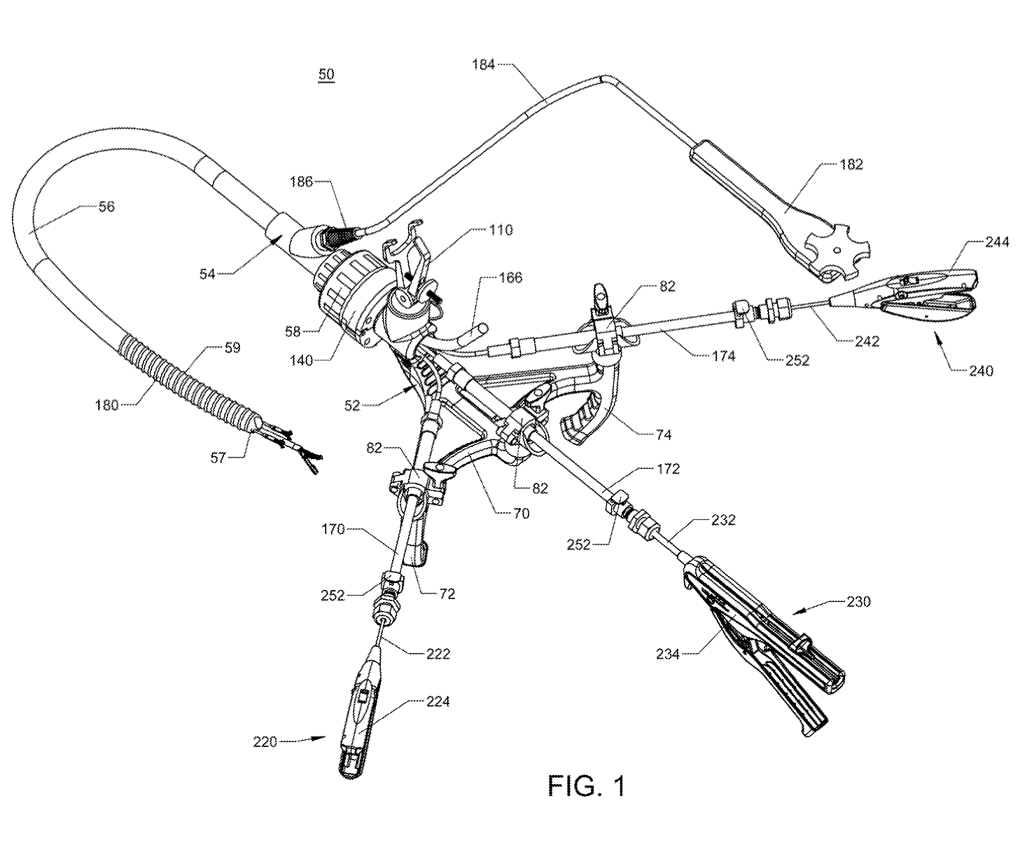

Turning to FIG. 1, an embodiment of a flexible surgery access and instrument management system 50 is shown. The system is used to perform diagnostic and therapeutic procedures in a minimally invasive manner. The system 50 includes an ergonomic and efficient user interface that allows the system to be used either in an endoscopic “hand held” condition or in a surgical “stand held” condition. In both conditions, the system provides the user with the capability to push, pull, and/or twist the insertion portion of the device for access and positioning in the human body. The system also provides the user with the ability to manage, park, govern, and/or secure any surgical instrument(s) that are used in association with the system.

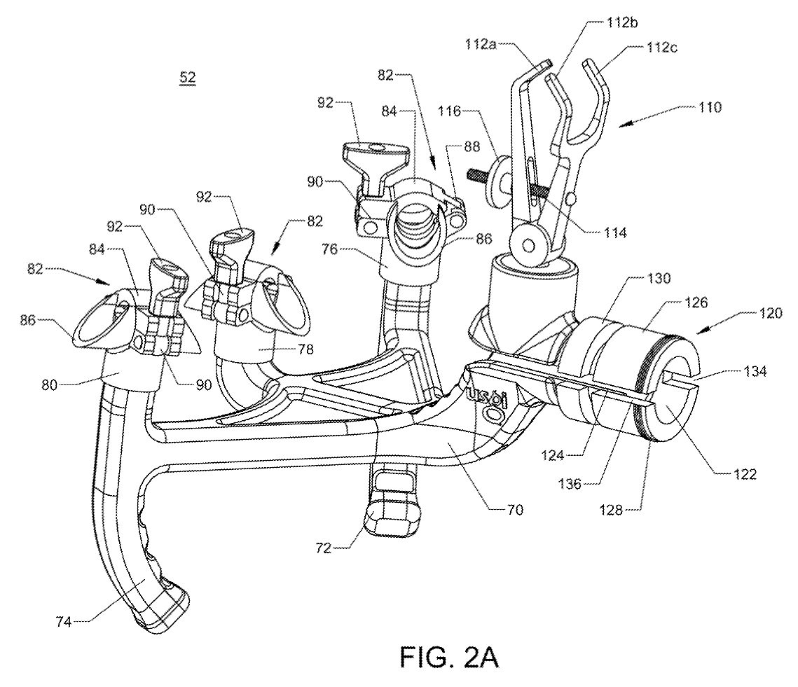

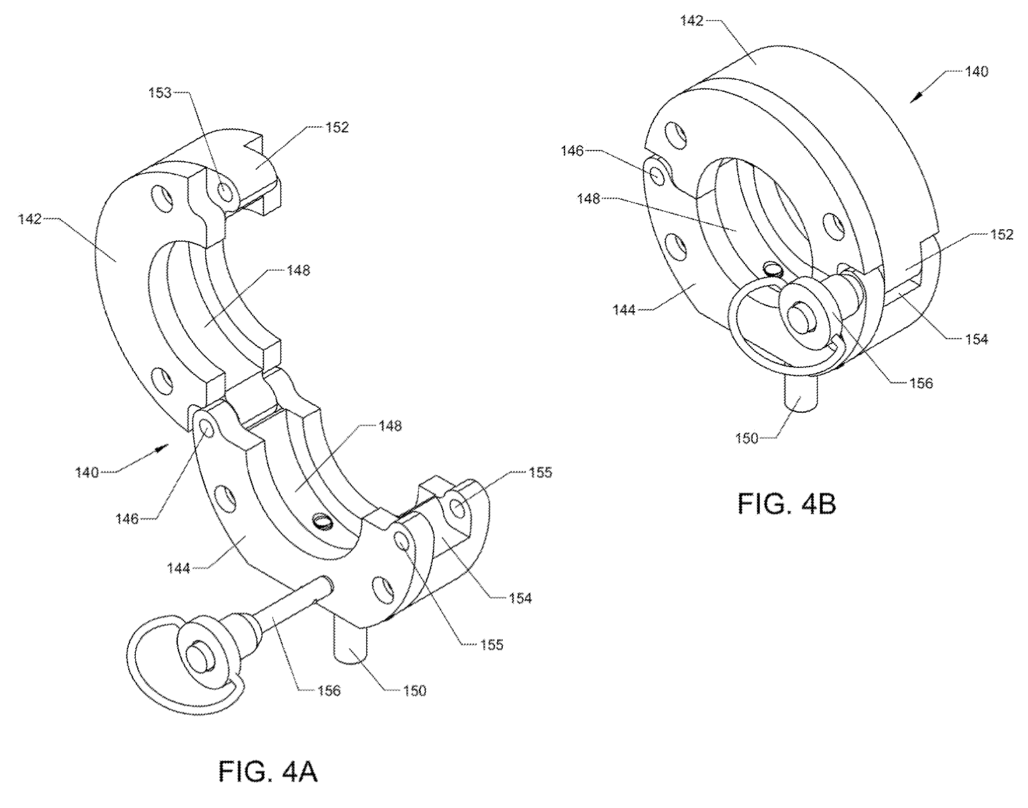

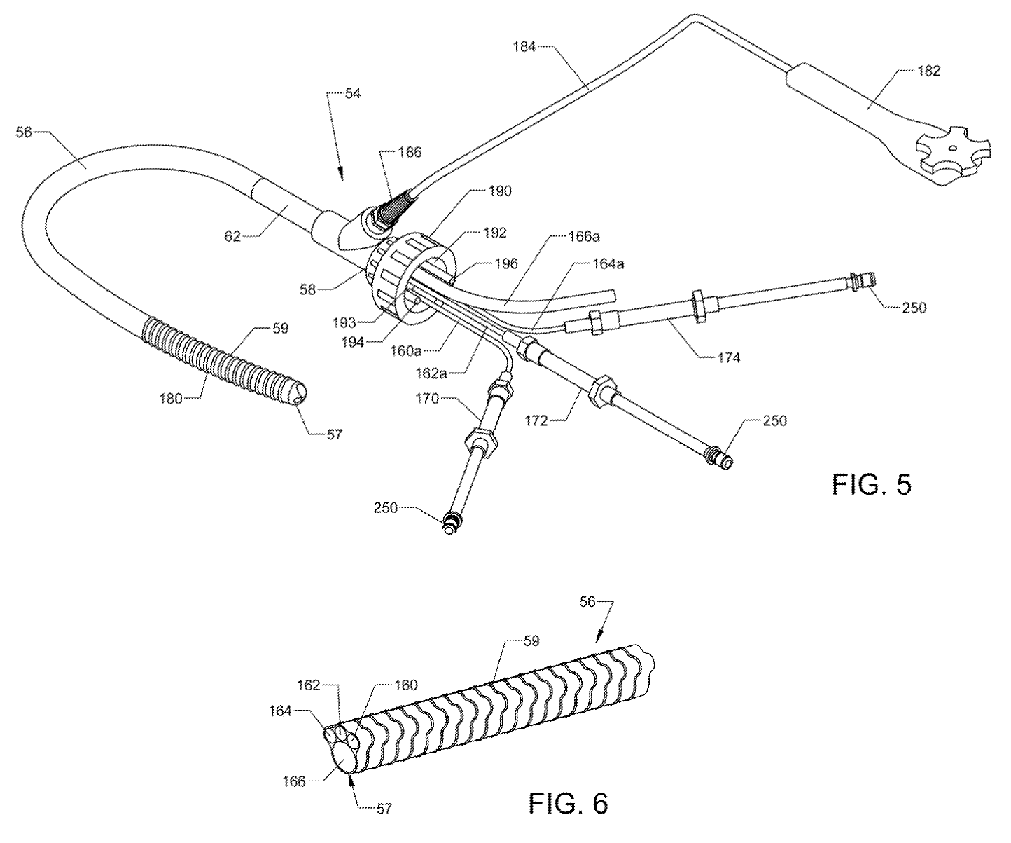

The embodiment of the system illustrated in FIG. 1 is resposable (reusable-in-part/disposable-in-part), having a reusable base unit 52 and a disposable insertion unit 54. The base unit 52 is cleanable and sterilizable, and provides a stable platform having a connection mechanism 120 (see FIGS. 2A-B) to which the insertion unit 54 is attached. The insertion unit 54 includes an elongated conduit 56 having one or more tubes 160, 162, 164 (see FIGS. 5-6) providing instrument passages, and a connection mechanism 58 adapted to selectively couple with the mating connection mechanism 120 provided on the base unit.

### Base Unit

An embodiment of the base unit 52 is shown in more detail in FIGS. 2A-B. The illustrated base unit 52 includes a frame 70 having a pair of handle grips 72, 74 extending below the frame. The handle grips 72, 74 are of a size, structure, and orientation to allow a user to grasp and hold or move the system with one or two hands when desired. In some embodiments, the frame 70 is attached to a separate instrument stand in a selectively rotating manner. Several of these embodiments are described below.

A plurality of tube supports 76, 78, 80 are located on the upper surface of the frame 70, where they are attached to or formed integrally with the frame. In each of the base unit embodiments shown in FIGS. 2A-B, the three tube supports 76, 78, 80 are placed in a single plane in a generally fanned-out alignment relative to the longitudinal working axis of the system, including a center tube support 78 that is generally aligned with the axis, and a left tube support 76 and right tube support 80 that are aligned to the left and right, respectively, of the longitudinal axis (as viewed by the user from the proximal end of the system). In other embodiments, the tube supports are located in multiple planes. The fanned out orientation has the result that the working instruments are not crowded at the proximal end of the system. In the embodiments shown, the left and right support tubes 76, 80 are placed symmetrically left and right of the center tube support 78 at approximately the width of a human waist. In some embodiments, the left and right tube supports 76, 80 connect tubes that provide working channels that terminate at the tip in a generally left and right location. In this fashion, instruments placed through the tubes are grasped in a comfortable location with the user's hands generally at the waistline. The center tube support 78 is in the middle and slightly proximally located relative to the left and right tube supports 76, 80, with the result that the frame 70 has a generally “trident” shape with a shortened center tube support 78 and the left and right tube supports 76, 80 aligned outwardly. The center tube 162 provides a working lumen that exits the distal end of the elongated conduit 56 in the center of the other two tubes 160, 164. In some embodiments, the center tube 162 provides a passage for a retraction tool, suction irrigation, scissors, or a ligation device, while the left 160 and right 164 tubes provide passages for a grasper, a dissector, and/or manipulation tools that arrange the anatomy conveniently for the instrument carried by the center tube 162. A particular example includes two graspers used to manage and tension a vessel via the opposing outer tubes 160, 164 and a scissors or clip that is passed through the center tube 162 between the secured graspers.

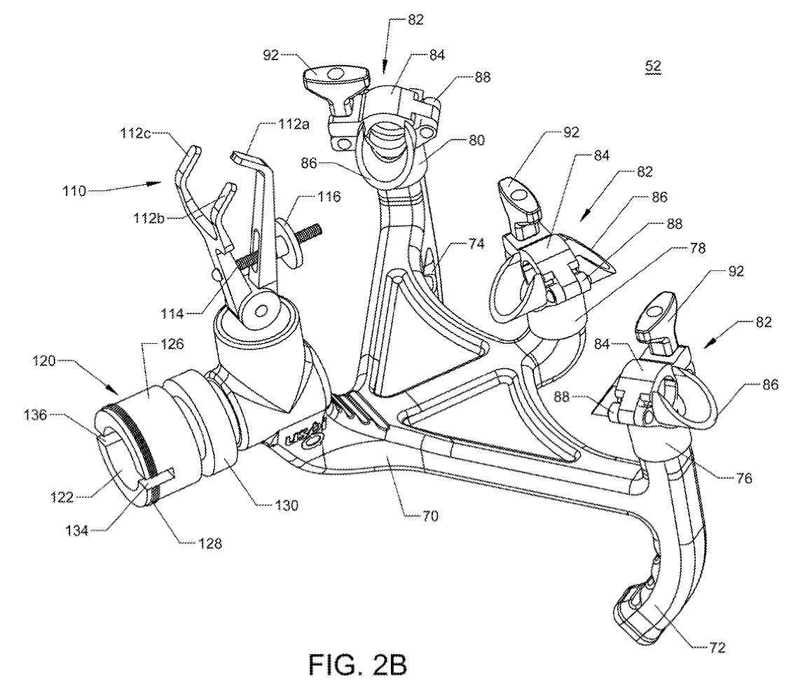

The tube supports 76, 78, 80 provide a mechanism for releasably attaching the proximal ends of the tubes 160, 162, 164 of the disposable insertion unit 54 to the base unit 52. There are several types of attachment mechanisms suitable for use as tube connectors, of which one collar-type embodiment is shown in the Figures. In the embodiment shown in FIGS. 2A-B, each of the three tube supports 76, 78, 80 includes a split collar clamp 82 having an upper collar portion 84, a lower collar portion 86, and a hinge 88 rotatably connecting the upper and lower collar portions. A pivoting screw 90 and wing nut 92 are positioned to engage and apply a compression force to the upper collar 84 after it has been rotated into place above the lower collar 86. In reference to FIGS. 3A-C, the split collar clamps 82 shown in FIGS. 2A-B are operated by pivoting the screw 90 away from the collar to allow the upper collar 84 portion to be hinged open. (See FIG. 3A). The proximal portion of one of the tubes of the disposable insertion unit 54 is then set between the open collar halves 84, 86 and then the halves are brought together to clamp onto the tube. (FIG. 3B). The pivoting screw 90 and wing nut 92 are then swung back in to a location where the wing nut 92 can then be advanced so as to abut the collars and apply a lockdown pressure. (FIG. 3C). In some embodiments, the tube 160, 162, 164 and collar 82 have mating features that dictate the linear and rotational position of the tube for clamping. For example, in some embodiments, the tube 160, 162, 164 includes a square collar at a specific location and the split clamp 82 has an internal receiving recess to accept the square collar. This mating relationship is useful for clamping the tube 160, 162, 164 at a specific location, and/or to prevent linear or rotational relative motion between the tube and the tube support. In some embodiments, the collar 82 has a more complex shape that allows the tube to be held in only a single orientation.

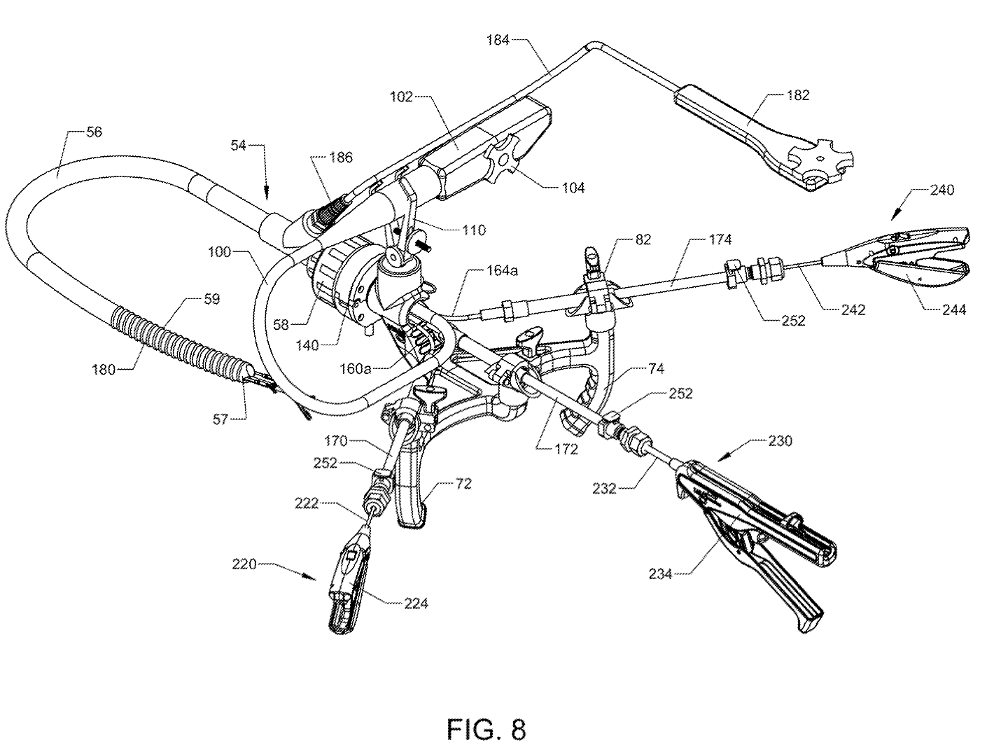

The frame 70 also accommodates an additional tube 166 and/or otherwise provides access into the elongated conduit 56 of the insertion portion for a flexible endoscope 100. In some embodiments, the endoscope tube 166 is retained in an endoscope tube support (not shown in the drawings). In other embodiments, the endoscope tube 166 is not received in a tube support. Instead, in those embodiments, the proximal portion of the endoscope tube 166 is generally flexible and is able to be moved along with the endoscope 100. In some of these embodiments, the frame 70 includes a clamp or holder 110 that releasably retains the handle 102 or other portion of the endoscope. See FIGS. 2A-B and FIG. 8. In the embodiment shown, the clamp or holder 110 includes a multiple-prong clamp having three prongs 112a-c and an adjustment mechanism including a threaded stud 114 and an adjustment nut 116. Those skilled in the art will recognize that other clamping and holding mechanisms would be suitable for retaining the endoscope 100 in place. In this manner, the endoscope 100 is selectively attached to the base unit 52.

The frame 70 includes a connection mechanism 120 for removably attaching the insertion unit 54 to the base unit 52. In some embodiments, the connection mechanism 120 is located and oriented so that the insertion unit 54 is retained in a position aligned with the longitudinal working axis of the system. The connection mechanism 120 includes a passage, lumen, or other opening 122 having a size and orientation allowing the tubes of the insertion unit 54 to be extended through and then fanned out for connection to the tube supports 76, 78, 80. In some embodiments, the frame 70 includes an opening 122 large enough for the tube bundles and tube terminations to pass therethrough. In other embodiments, the frame 70 is separated into two or more pieces that are combined or attached around the tube bundles. In still other embodiments, the frame 70 includes a pair of opposed hinged members so that the frame may be unlocked, opened, loaded, and then closed around the tube bundles. In still other embodiments, a slot 124 is formed in a portion of the connection mechanism 120 providing access to the opening 122 such that the lumen bundles may be slid into the opening 122 for loading.

For example, as shown in FIG. 2A, an embodiment of a connection mechanism 120 is formed on the distal end of the frame 70 of the base unit. The frame 70 includes a central lumen 122 extending along the longitudinal axis of the system and having a size that accommodates the tubes extending through the elongated conduit 56. The connection mechanism 120 includes a flange 126 having threads 128 formed on its exterior surface. A slot 124 is formed in the frame and extends from the distal end of the frame to the proximal end of the central lumen 122. The slot 124 provides access through the side of the frame 70 and into the center lumen 122. The manner in which the disposable insertion unit 54 is removably attached to the base unit 52 through the connection mechanism 120 is described more fully below in the description of the insertion unit 54, below.

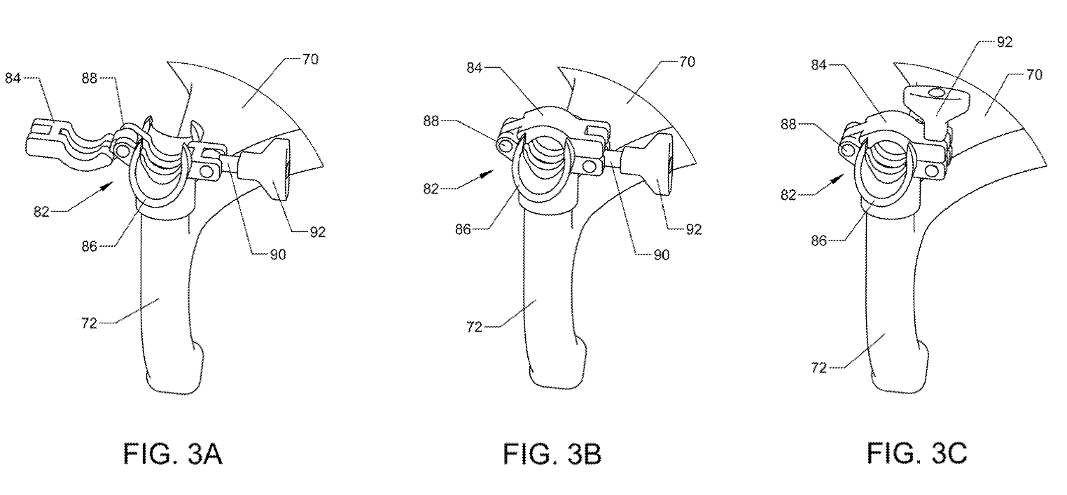

In the embodiment shown in FIG. 2A, the base unit 52 also includes a disk 130 that is attached to or formed integrally with the frame at a location that is just proximal of the flange 126 of the connection mechanism. The disk 130 provides an external feature that is of a size, shape, and orientation to be engaged by a circular rotating clamp 140, such as the clamp 140 shown in FIGS. 4A-B. Additional details relating to the circular rotating clamp 140 and the disk 130 to which the rotating clamp is adapted to rotatably attach are provided in U.S. patent application Ser. No. 12/138,348, filed Jun. 12, 2008, and incorporated by reference above. The rotating clamp 140 includes a top half 142 and a bottom half 144 connected by a hinge 146, with each of the top half 142 and bottom half 144 comprising a semi-cylindrical member having an interior groove 148. The interior groove 148 is of a size and shape to rotatably engage the disk 130 formed on the frame 70 of the base unit 52. As a result, when the rotating clamp 140 is placed over the disk 130, the rotating clamp 140 forms a housing that is able to rotate around the longitudinal working axis of the access system while being supported by the disk 130. A tab 152 having a through-hole 153 is formed on the portion of the top half 142 of the clamp opposite the hinge 146. A mating groove 154 is formed on the portion of the bottom half 144 of the clamp opposite the hinge 146, with a pair of through-holes 155 formed in the sidewalls of the groove 154. A pin 156 is provided having a size to slide through the through-holes 153, 155 in order to maintain the top half 142 and bottom half 144 in the closed position, as shown in FIG. 4B.

In some embodiments, such as shown in FIGS. 4A-B, a post 150 or other engagement member is formed on the circular rotating clamp 140. The post 150 or other engagement member provides an interface for engagement of the rotating clamp 140 to a surgical instrument stand, support arm, or other fixed member. These stands and other fixed members are readily available in a surgical suite and are often used to hold laparoscopes or liver retractors in position. The instrument stands typically include a single or series of rigid metal beams connected in a single or multiple pivoting manner so that the end most portion may be positioned freely relative to the patient. The instrument stands are usually able to be locked in a specific position with an activation knob. Some instrument stands are in the form of a metal gooseneck. Other instrument stands are in the form of one or more pneumatic cylinders that lock and unlock. Still other instrument stands have weighted counterbalances so the held instruments “float” in a zero or reduced weight fashion. Still other instrument stands remain in a semi-locked condition and are malleable for positioning. Still other instrument stands have no motion and have a single fixed configuration.

As described above, in the embodiment shown in FIGS. 2A-B, the disk 130 is attached to or formed integrally with the frame 70 of the base unit. The housing of the rotating clamp 140 (FIG. 1, FIGS. 4A-B) includes an interface, such as a post 150 or other connection to the instrument stand. In some embodiments, the rotational connection of the flexible surgery access system 50 to the stand is configured around the center of gravity of the completely assembled system so that the system is not top heavy and inclined to tip unintentionally. Alternatively, the rotational attachment is placed above the center of gravity so that the system 50 tends to stay “righted” unless actively rotated. Rotation of the system has been found to have a very significant usefulness during surgical procedures. In addition, as discussed below, the attached insertion unit 54 is connected so that it is rotated when the handle grips 72, 74 and frame 70 of the base unit 52 are rotated. This is an effective way to move instruments in the surgical field. For example, if the distal region of the insertion unit 54 is slightly steered, then rotation of the frame 70 causes the insertion unit 54 tip to move in a smooth arc. This movement, in turn, causes movement in a smooth arc of any tools disposed within the tubes of the insertion unit 54. The rotational motion is simple for a user to apply and is intuitive. The user grasps a graspable portion on the frame 70 or simply applies a rotation thru the instruments contained in the system (more description on this subject is provided later in the disclosure).

In some embodiments, the rotating clamp 140 includes one or more stops to limit the extent of rotation so as not to rotate beyond a specific point (e.g., 45 degrees). In other embodiments, the rotating clamp 140 includes a lock or break to stop rotation at a specific or multiple locations. In still other embodiments, the rotating clamp 140 includes a damper or base friction to keep the motion free form sudden jerks or uncontrolled motion. In still other embodiments, the rotating clamp 140 includes or is attached to a linear slidable connection so that the frame 70 may be used to move the system 50 in and out relative to the incision.

### Insertion Unit

The insertion unit 54, illustrated in FIG. 5, includes an elongated conduit 56 having a tip 57 at its distal end. The proximal end 62 of the elongated conduit is attached or formed integrally with a connection mechanism 58 that is constructed to be removably attached to the mating connection mechanism 120 included with the base unit 52. A plurality of tubes 160, 162, 164, 166 extend through the elongated conduit 56, terminating at the distal tip 57. The proximal ends 160a, 162a, 164a, 166a of the tubes extend proximally out of the proximal end of the elongated conduit 56 and beyond the connection mechanism 58. In some embodiments, the insertion unit 54 also includes telescoping tube terminations 170, 172, 174 which are described more fully below, at the proximal ends of the tubes. In some embodiments the insertion unit has a controllable steering section 180 that provides the user with the ability to curl the elongate conduit 56 of the insertion unit 54 in one or multiple planes. The controller 182 of this function is preferably included with the insertion unit 54. As noted above, in some embodiments the insertion unit 54 is disposable so that the intimate contact and/or hard to clean portions of the system 50—such as the outer surface of the elongated conduit 56 and the internal tubes 160, 162, 164—are limited to single use.

The elongated conduit 56 has a diameter and length appropriate to provide access for performing the required diagnostic or therapeutic procedure. For many such applications, the elongated conduit 56 is 20 [mm] or less in crossing profile and has a length of 25 to 150 [cm]. In particular, the transgastric access requires the longest length and smallest diameter due to its location deep within the body. Other access points may require a shorter length and/or may not require as small a diameter in cross-section.

Alone or jointly with a controllable steering section 180, in some embodiments, the insertion unit 54 includes a distal region 59 and distal tip 57 that are pliable. See FIG. 6. In those embodiments, it is intended that the endoscope 100 extending through an endoscope tube 166 of the elongated conduit 56 be steered using its normal steering controls 104 and that the endoscope 100 will move the distal region 59 and pliable tip portion 57 of the elongated conduit 56 with it. This mode of operation is referred to as “slave steering” or “passive steering.” In these embodiments, the endoscope 100 is relatively large compared with the included tool tubes 160, 162, 164 and intended tools. For example, typically the endoscope 100 has an outer diameter of about 10 [mm] and the tools have an outer diameter within a range from 2-5 [mm]. The endoscope 100 has enough steering power to move the elongate conduit 56 sleeve and the tools or instruments extending through the tubes 160, 162, 164.

In some embodiments, a combined steering capability is provided by having a pliable distal region 59 and distal tip 57 that is able to follow the steering of the endoscope 100, and a steering section 180 of the elongated conduit 56 that is under a separate control. For example, in some embodiments, the distal 5-10 [cm] region of the elongated conduit 56 is slave steered by the endoscope 100. The next 5-15 [cm] region located adjacent to and proximally of the distal region has an active “lift” in one, two, three, four, or more planes that is under control of the steering control 182 of the insertion unit 54. The two steering sections and steering capabilities combined allow the distal region 59 and tip 57 to form a multitude of complex curves for positioning as desired by the user. For example, the elongated conduit 56 of the insertion unit 54 is able to form an “S” or “U” shape. The “S” shape is suitable for straight on approaches where the distal region 59 and tip 57 is lifted and directed down onto a target site of interest. The “U” shape is suitable for retroflex work.

In the embodiments described, the insertion unit 54 includes a connection mechanism 58 for connecting the insertion unit 54 to the base unit 52. In several embodiments, the connection mechanism 58 is provided with features that allow the user to selectively attach the insertion unit 54 in multiple rotational orientations. For example, in some embodiments, the insertion unit 54 is able to be connected at a 12 o'clock or 6 o'clock orientation so that the tube bundles 160, 162, 164 exit the elongated conduit 56 at the distal tip 57 in a selected alignment relative to the base unit 52.

In the embodiment shown in FIG. 5, the connecting mechanism 58 of the insertion unit includes a cylindrical cap 190 having threads 192 formed on an interior surface that are adapted to mate with the threads 128 on the external surface of the connection mechanism 120 of the base unit 52. A sealing plate 193 is located within the cap 190 and is attached to or formed integrally with the proximal end of the elongated conduit 56. The sealing plate 193 includes a center passage through which the insertion unit tubes 160, 162, 164, 166 extend proximally. The cap 190 is able to rotate relative to the elongated conduit 56 and the sealing plate 193. A pair of alignment pins 194, 196 are fixed to the proximal face of the sealing plate 193 such that the user is able to line up the alignment pins 194, 196 with mating slots 134, 136 included on the base unit connecting mechanism 120. In this way, the user is able to select a desired orientation to achieve, for example, either a 12 o'clock or a 6 o'clock alignment of the distal tip 57 relative to the base unit 52.

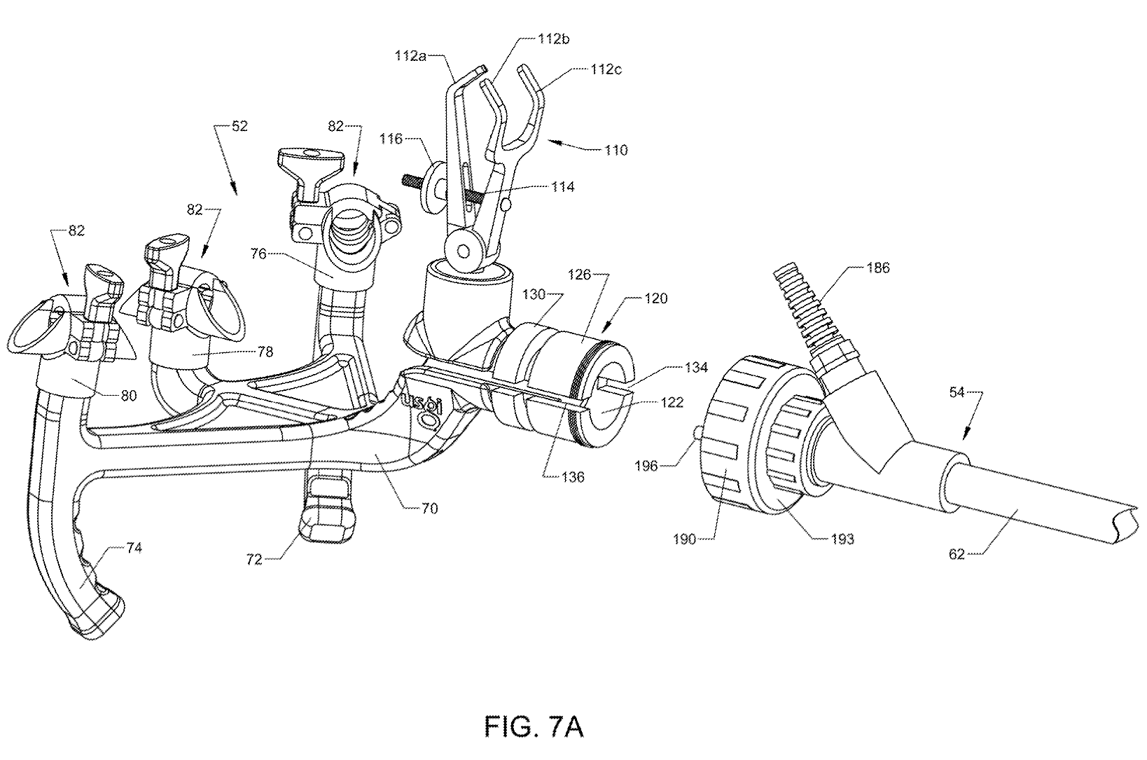

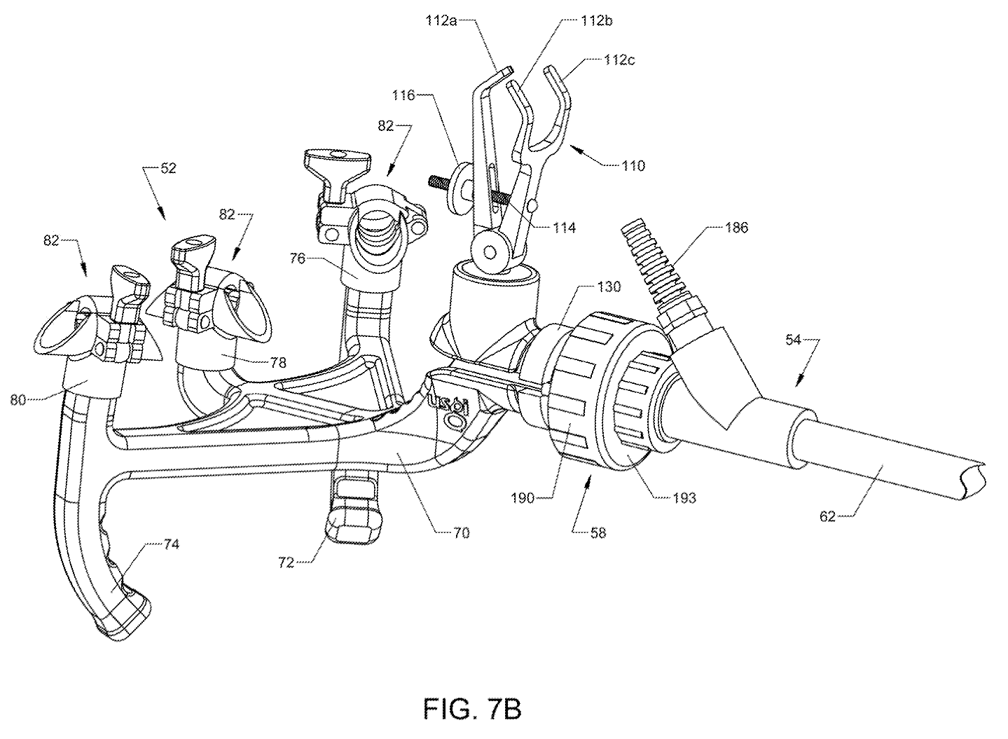

The operation of the illustrated connection mechanism 58 will now be described. First, the insertion unit 54 and base unit 52 are brought near to each other. (See FIG. 7A). The tubes 160, 162, 164, 166 of the insertion unit 54 are placed through the slot 124 on the frame of the base unit and into the center lumen 122. (For clarity, the tubes 160, 162, 164, 166 of the insertion unit 54 are not shown in FIGS. 7A-B). The sealing plate 193 of the insertion unit 54 is placed against the distal opening of the center lumen 122 of the base unit while inserting the alignment pins 194, 196 into the mating alignment slots 134, 136 in the alignment desired by the user. The cap 190 of the insertion unit connection mechanism 58 is screwed onto the flange 126 of the base unit connection mechanism 120 until snug. (See FIG. 7B). The insertion unit tubes 160, 162, 164 are then placed into and secured in place by the tube supports 76, 78, 80. (See FIG. 1). Then, optionally, the circular rotating clamp 140 housing is brought over the disk 130 on the frame 70 of the base unit 52 and clamped shut. (See FIG. 1). In some embodiments, the steering controls 182 of the insertion unit 54 are on a fixed mounting that is on the insertion unit 54 and are manipulated by the user from that location. In other embodiments, the steering controls 182 of the insertion unit 54 have a fixed arm to bring the control 182 into a location on or near the handle grips 72, 74 of the base unit 52 that is more convenient for the user. In the embodiments shown in FIGS. 1 and 5, the steering controls 182 are on a flexible tether 184 attached to the proximal end of the elongated conduit 56 via a strain relief 186. The steering controls 182 may be handled independently by the user or an assistant, clamped along side the endoscope 100 in the endoscope clamp 110, attached at another clamping location on the base unit 52, or placed in another location at the user's discretion. In the illustrated embodiment, the steering controls 182 include a hook and loop (i.e., velcro) surface that engages a mating hook and loop surface on the bottom of the frame so that the steering controls are in a convenient location near to the user.

In an alternative embodiment, a bayonet-type connection mechanism is used, in which one or more engagement pins or tabs on one of the connection mechanisms (e.g., either the insertion unit connection mechanism 58 or the base unit connection mechanism 120) engage mating slots or grooves on the other connection mechanism, and then the connection mechanisms 58, 120 are rotated relative to each other to lock the connection mechanisms together. Other alternative connection mechanisms are also suitable.

As discussed above, in some embodiments the insertion unit tubes 160, 162, 164 terminate at their proximal ends 160a, 162a, 164a in simple fittings that are releasably retained in the tube supports 76, 78, 80. In other embodiments, the proximal ends 160a, 162a, 164a of the tubes include a telescoping feature that provides the system with the capability of inserting and retaining flexible endoscopic tools and instruments in an advantageous telescoping arrangement within the system. In still other embodiments, the proximal ends 160a, 162a, 164a of the insertion unit tubes include a rigid tube section of about 5 [cm] to about 15 [cm] in length. The rigid tube sections of the tubes are adapted to be used with instruments 220, 230, 240 having a rigid body section 222, 232, 242 adjacent to the instrument handle 224, 234, 244 such that each of the rigid tube sections 200, 202, 204 and its respective rigid body section 222, 232, 242 engage in a supported telescoping relationship. In some of those embodiments, a gas/fluid seal is included on the interior of each of the rigid tube sections. Additionally, in some embodiments a port for the introduction of insufflation gas or lubrication fluid is provided on the rigid tube section. For example, a “Y” port with a luer connection on each lumen termination may be provided.

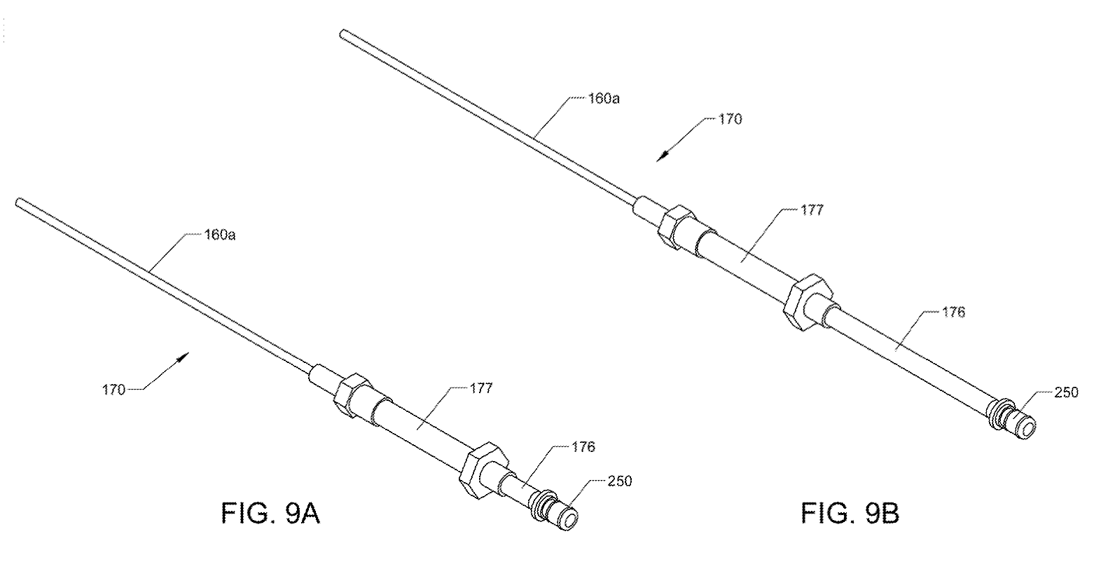

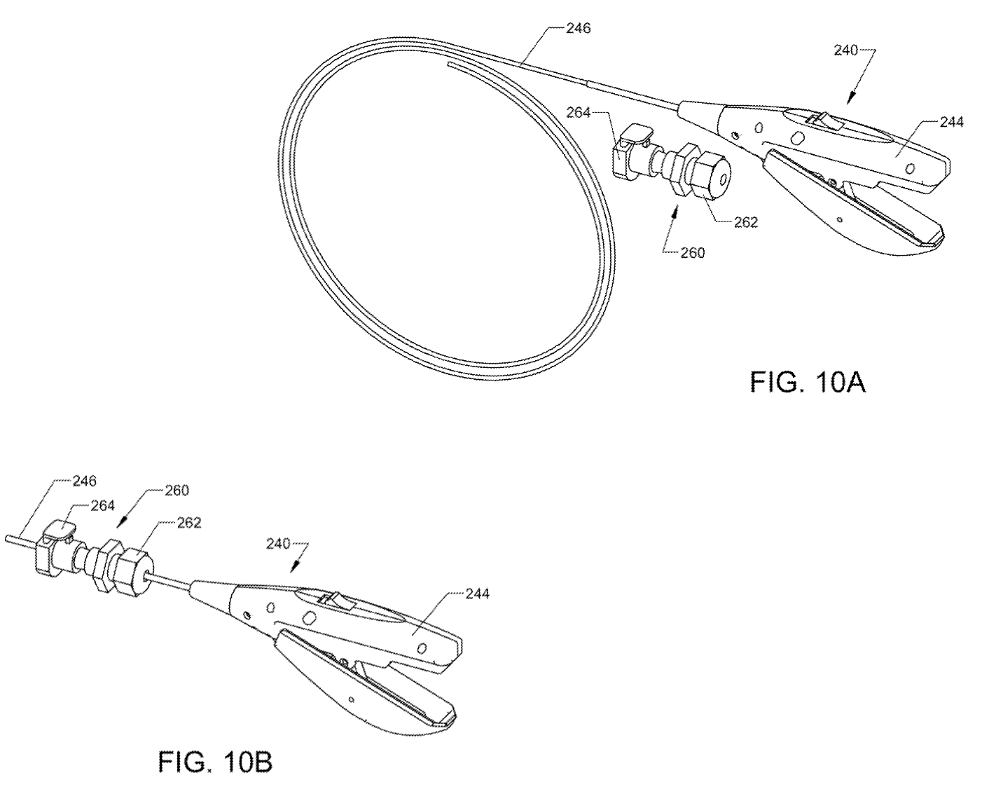

In alternative embodiments, shown in FIGS. 1, 5, and 8, the proximal insertion tube terminations include a pair of telescoping rigid tube sections 170, 172, 174. In these embodiments, the telescoping functionality of the rigid tube sections 170, 172, 174 eliminates the need for the introduced instruments 220, 230, 240 to have a rigid shaft section 222, 232, 242, thereby providing the capability to use fully flexible shafted instruments within the system. (See FIGS. 10A-B). For example, in some embodiments, the proximal rigid tube section 170, 172, 174 includes inner 176 and outer 178 close fitting tubes. (See FIGS. 9A-B) In those embodiments, the inner tube 176 has a very close fluid and gas tight fit (similar to a glass syringe) or, alternatively, the inner 176 or outer tube 178 includes a gasket or o-ring sealing component to allow relative motion with a gas/fluid seal.

In the telescoping tubes 170, 172, 174 embodiments, the ends of the outer tubes each include a fitting that allows the inner tubes to slide in the outer tube but not slide free of the outer tube. The inner tubes each have a connector 250 that reliably locks to a mating connector 252 on the shaft of the instrument 220, 230, 240 introduced into the system through the respective insertion unit tube. The interlocking connectors 250, 252 thereby provide a sealed and slidably supported relationship between the instrument and the insertion unit tube. Several suitable male/female interlocking connectors are available, such as an open flow coupler, quick connect coupler, CPT connector, or the like. In other embodiments, the interlocking connector includes a non-standard mechanism or sensor (e.g., mechanical, electrical, etc.) that requires a particular non-standard mating mechanism in order to allow the system to function.

The interlocking of an instrument 220, 230, 240 or tool to a telescoping rigid tube section 170, 172, 174 of the insertion unit provides a mechanism for limiting the depth or withdrawal length that the tool is able to be moved. This has the advantage of preventing an instrument from inadvertently sliding out of the system. In other embodiments, additional features may be added to the telescoping rigid tube sections 170, 172, 174. For example, in some embodiments, a variable friction or locking feature is incorporated to dampen or prevent linear or rotational motion of the telescoping sections. An example of such a locking feature includes pins and slots formed on mating surfaces of the telescoping components and arranged to allow for only predetermined motions. In other embodiments, the telescoping sections 170, 172, 174 include one or more springs oriented to automatically position an instrument or tool back to a default position when the user removes his or her hand from the instrument or tool. In still other embodiments, the telescoping sections 170, 172, 174 are configured with tactile or visual indicators that indicate the position of a portion of the tool or instrument relative to the distal tip 57 or other portion of the flexible surgery access system.

In still other embodiments, an adapter 260 is attached to an instrument or tool 240, with the adapter 260 being configured to mate with a connector 250 or other entry point into a tube of the system 50. One example of an adapter 260 is shown in FIGS. 10A-B. The adapter embodiment is an iris clamp 262 attached coaxially with the interlock 264. It can be slid and fixed to a tool shaft 246 to provide the connection to the telescoping tube 170, 172, 174 of the flexible surgery access system 50.

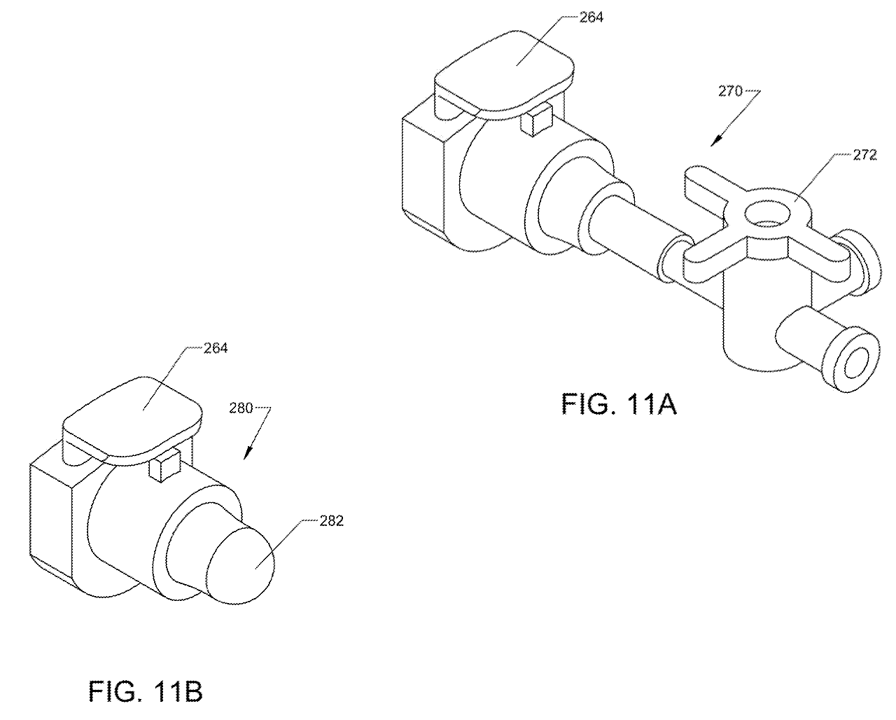

In alternative embodiments, adapters are provided with additional functionality. For example, in the embodiments shown in FIGS. 11A-B, a first adapter 270 embodiment includes an injection or gas/liquid flow attachment 272 attached coaxially with the interlock 264, and a second adapter embodiment 280 includes no outlet so as to create a cap 282 for a telescoping tube 170, 172, 174.

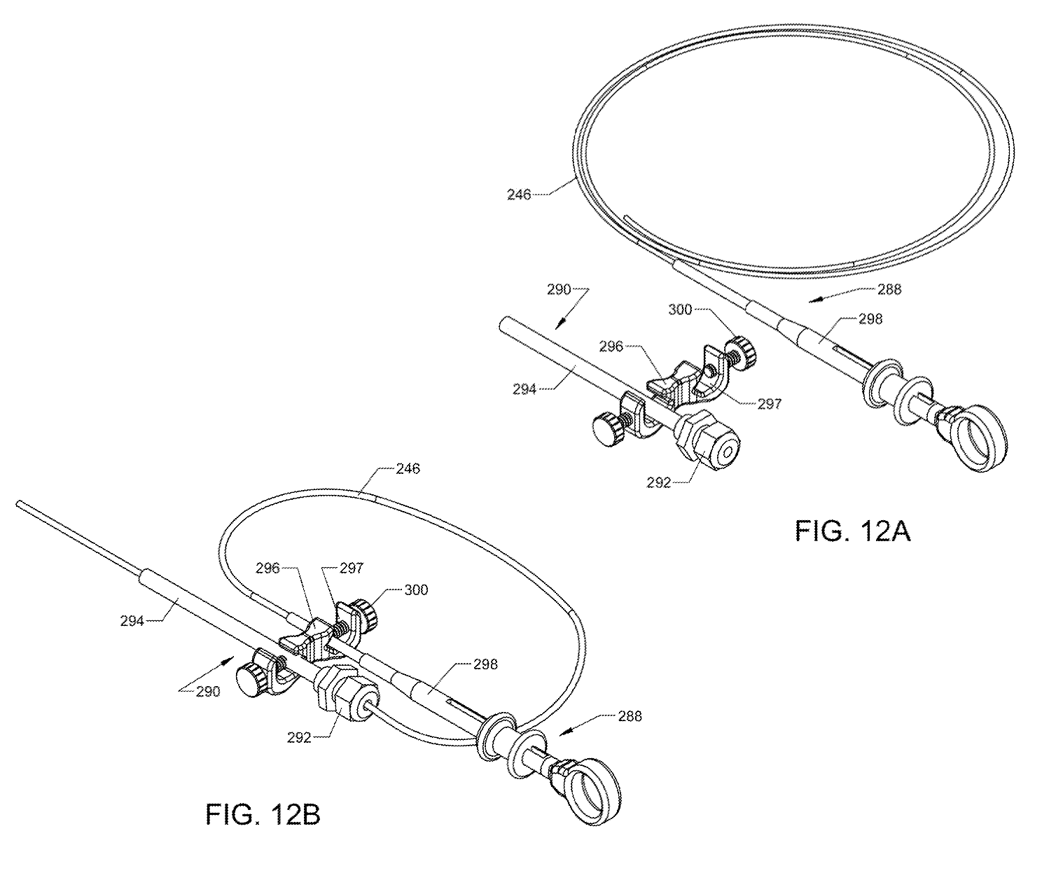

Still other examples of adapters suitable for use with the flexible surgery access system include features that facilitate the use of a conventional fully flexible shaft instrument or tool within a system that is not provided with telescoping tubes 170, 172, 174. For example, as shown in FIGS. 12A-B, a flexible elongated endoscopic biopsy instrument 288 typically includes a grasping cups tip of 2.8 [mm], an elongated coil body of 100-200 [cm], and a finger ring handle. An alternative adapter 290 embodiment includes a touhy borst type iris valve 292 attached in line to a rigid tube 294. The adapter 290 includes a plate, clamp 296, or other member that is attached to or formed integrally with the iris valve 292 and tube 294 assembly and that also includes a hole, slot 297, or other engagement portion adapted to receive the instrument ring handle 298. A set screw 300 is provided to lock the instrument in place relative to the adapter 290. The excess length of the flexible shaft 246 is coiled and the instrument head and working length of the shaft 246 is fed through the iris lock-down feature 292. Then, the instrument 288 may be inserted into a tube 160 regardless of whether the tube 160 includes a telescoping tube termination 170, 172, 174. In this way, the fully flexible shaft endoscopic instrument gains some of the advantages of the slidably supported interface described above.

The flexible surgery access systems described herein include several features that improve the ability of the user to correct or modify the images viewed through the endoscope such that the images are more intuitively associated with the movement and operation of the instruments being used with the system. For example, the ability to translate and rotate the endoscope 100 relative to the instrument tubes 160, 162, 164 is beneficial. During some procedures, the endoscope 100 is advanced ahead of the tubes 160, 162, 164 and tools 220, 230, 240 to obtain additional views of the surgical space. The endoscope 100 thereby performs a “scouting” function independently of the system to perform diagnostic work before the advancement of the therapeutic instruments. The endoscope 100 may be extended and retroflexed to view the tubes 160, 162, 164 and instruments 220, 230, 240.

In addition, the endoscope 100 may be twisted within its tube 166 relative to the other tubes. This allows the user to align the endoscope 100 with the true “Up-Down” horizon. By twisting the endoscope 100 or, alternatively, the surrounding lumen bundles 160, 162, 164, the user is able to align the instruments 220, 230, 240 at any orientation around the periphery of the endoscope 100. In this manner, the user is able to advance the instruments 220, 230, 240 into the working and visual space at any selected alignment relative to the endoscope 100.

As discussed above, the system described herein has the capability to pre-align the tube bundle 160, 162, 164 relative to the scope 100 in any desired position by using the alignment pins 194, 196 and alignment slots 134, 136 of the connection mechanisms 58, 120. This capability allows the user to adjust the system alignment prior to use depending on if the system will be used in a fully forward, fully retroflexed, or any other orientation. For example, in a forward viewing orientation it may be desirable to set up the system so that the working tube bundle 160, 162, 164 is positioned below the endoscope 100. This creates a more natural “head above hands” view. However, if the system is intended to be used in a retroflexed orientation, the tubes 160, 162, 164 in the same alignment as in the forward viewing orientation would end up in an orientation above the endoscope 100 once the endoscope 100 is rotated to correct the view after retroflexing the elongated conduit 56. For the retroflexed case, the tube bundle 160, 162, 164 may be pre-aligned so as to be above the endoscope 100 in the forward configuration, so that the tubes 160, 162, 164 end up below the endoscope 100 in the retroflexed configuration, after endoscope correction.

In addition, in the retroflexed orientation, the endoscope 100 must be rotated 180 degrees to establish a true “up-down” horizon. If the endoscope 100 is not rotated in this way, the resulting image provided by the endoscope 100 has an orientation in which “the ceiling becomes the floor.” However, when the endoscope 100 is rotated to create a true “up-down” orientation, then the right-left visual representation of instruments 220, 230, 240 exiting in the working space becomes reversed relative to the user end—i.e., moving the left handle instrument forward results in the instrument on the right side of the visual field moving forward. To correct for this result, it is useful to switch the proximal ends 160a and 164a of the tubes at the user end to their opposite sides. The selectively removable tubes held in the tube supports 76, 78, 80 provides the user with this functionality.

In summary, there are three physical corrections that are required to reset the visual field to a “true” image when the system is used in retroflexion: 1) pre or re-aligning the tube bundle 160, 162, 164 so that the instruments exit the tubes at a position “below” the endoscope 100; 2) correcting the true up-down image by rotating the endoscope 180 degrees within the endoscope tube 100; and 3) correcting for true right-left orientation after the endoscope rotation by switching the tube sides 160a and 164a on the user end.

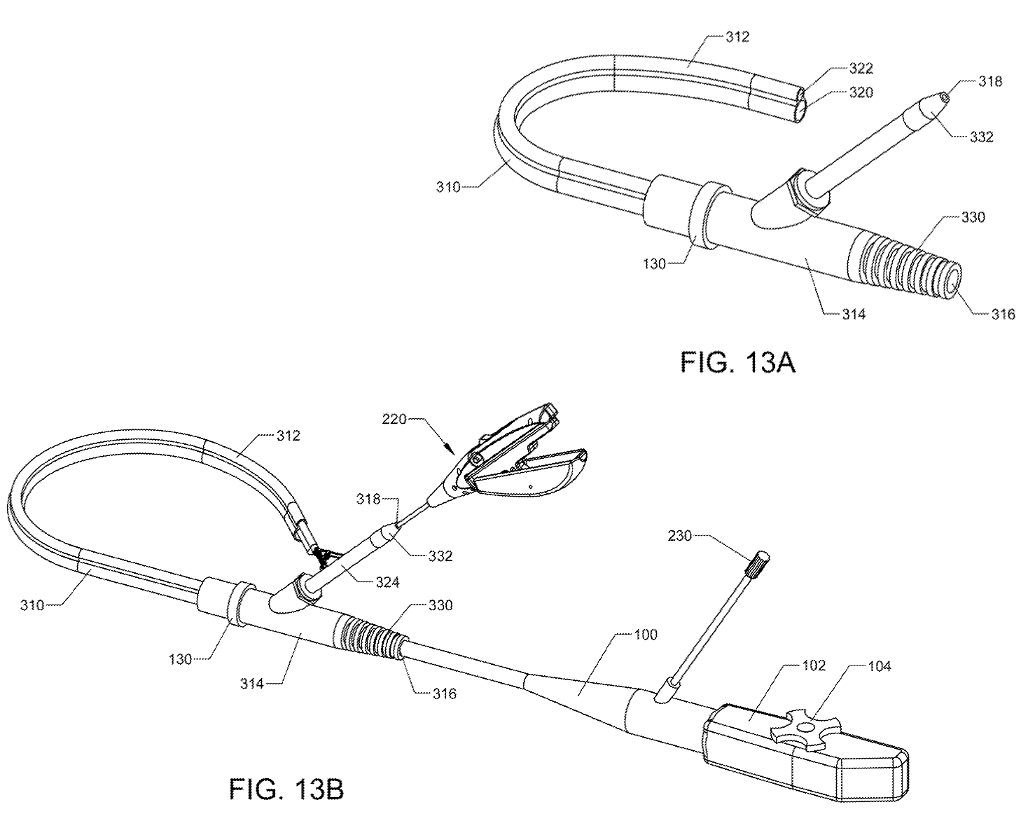

Another embodiment of a flexible surgery access system is shown in FIGS. 13A-B. The system includes an elongated flexible sleeve 310 that accepts an endoscope 100. The sleeve 310 includes a single internal tube 320 or multiple internal tubes 320, 322 located internally of the sleeve 310 or attached to the exterior of the sleeve 310. The distal region 312 of the sleeve is sufficiently flexible/pliable to be slave steered by the endoscope 100. The resulting multilumen sleeve 310 allows a user to use accessories (e.g., tools, instruments) with an endoscope 100 that cannot otherwise be placed through the working channels of the endoscope 100. The endoscope 100 has size limitations on the diameter of tools that fit through its working channels, and has a limited number of working channels. For example, tools that are accepted by conventional endoscopes are typically limited to having an outer diameter of 2.8 [mm] or less with relatively short rigid sections on the end effectors so that they may pass through the acute bend in the handle portion of most endoscope working channels.

The handle 314 includes seals 330, 332 for sealing around both the endoscope 100 and any instrument 220, 230, 240 used with the system. In some embodiments, the seals 330, 332 are zero seals that provide a sealing function regardless of whether the endoscope 100 or instrument 220, 230, 240 are in place. In other embodiments, the endoscope 100 and instrument 220, 230, 240 are sealed with a touhy borst type connector that also functions to maintain the linear and rotational position of the instrument or endoscope. In some embodiments, the handle 314 includes ergonomic features, including a grip member. In the embodiment shown, the handle 314 also includes a disk feature 130 adapted to be rotatably received within a circular rotating collar 140 so that the handle 314 and system may be selectively rotationally supported in a fixed stand.

In the embodiment shown in FIG. 13B, the endoscope 100 is introduced in a relatively straight on-axis approach through an endoscope port 316 with the accessories introduced through an instrument port 318 orientated at an acceptable angle. In alternative embodiments, the endoscope 100 is placed through the angled channel and the instruments 220, 230, 240 through a straight channel. In still other embodiments, all of the channels converge into the handle 314 with a slight angle.

Also in the embodiment illustrated in FIG. 13B, the access system is provided with a sleeve 310 having an 18-20 [mm] crossing profile. The sleeve 310 includes two lumens: a first lumen 320 having an inner diameter of 10-12 [mm] accommodates a gastroscope 100 (e.g., Olympus GIF H 180), and a second lumen 322 has an inner diameter of 6-7 [mm] to accommodate a tissue approximation and securing device 220 (e.g., USGI Medical g-Prox®). In alternative embodiments, the second lumen 322 accommodates another type of accessory, such as graspers, scissors, needles, suction/irrigation probes, electrocautery probes, clip appliers, or other endoscopic devices. An additional tissue grasper or retractor 230 is provided having a size that allows its use through the working channel of the endoscope 100. In the embodiment shown, the tissue grasper 230 is a g-Lix™ helical tissue grasper (USGI Medical Inc., San Clemente, Calif.). The handle 314 includes a touhy borst fixture to seal and lock onto the endoscope. The accessory lumen 322 extends from the handle 314 with an elongated, rigid portion 324 to slidably support the accessory 220. The accessory lumen 322 also includes a touhy borst valve/seal. In an alternative embodiment, the sleeve 310 also includes an additional lumen having a diameter of about 2-3 [mm] to provide CO2 (or other) insufflation. A circular rotating clamp 140 (not shown in FIG. 13B) rotatably supports the system in a fixed stand.

The devices described herein are suitable for use in many diagnostic and therapeutic procedures performed endoscopically, laparoscopically, endolumenally, translumenally, or any combination of the above. Examples of such procedures include endolumenal treatment of obesity (see, e.g., United States Provisional patent application Ser. No. 12/409,335, filed Mar. 23, 2009, hereby incorporated by reference), revision of obesity procedures (see, e.g., U.S. patent application Ser. No. 11/342,288, filed Jan. 27, 2006, hereby incorporated by reference), treatment of gastroesophageal reflux disease (GERD) (see, e.g., U.S. patent application Ser. No. 11/290,304, filed Nov. 29, 2005, hereby incorporated by reference), gastrotomy closure procedures (see, e.g., U.S. patent application Ser. No. 11/238,279, filed Sep. 28, 2005, hereby incorporated by reference), wound closure, fistula repair, cholecystectomy, appendectomy, transvaginal procedures, transrectal procedures, transgastric procedures, single port access procedures, and others. Additional examples of procedures are described in the other patent applications incorporated by reference herein.

The devices, systems, and methods of the present invention have been described herein with respect to certain exemplary and/or preferred embodiments. Certain alterations or modifications are also included within the scope of the invention. For example, and without limitation, the foregoing description includes descriptions of embodiments of flexible surgery access systems having a reusable base unit and a disposable insertion unit. In alternative embodiments, the base unit has a construction that renders it suitable for disposal after a single use, and the insertion unit is sterilizable such that it may be reused. In still other embodiments, the insertion unit is fixedly attached to the base unit and the entire flexible surgery access system is either reusable or disposable. Still other variations are possible. The embodiments described are offered as illustrative, and not limiting, on the scope of the present invention.

What is claimed is:

1. An endoscopic instrument management system, comprising:
an insertion unit including an elongated conduit defining a first lumen and a plurality of flexible tubes extending through at least a portion of the first lumen;
a base unit having at least one handgrip and having a plurality of tube supports, each tube support being adapted to selectively engage a proximal portion of at least one of the plurality of flexible tubes of the insertion unit, the base unit also having a flange defining a second lumen and having a slot with a size sufficient to allow passage of the plurality of flexible tubes of the insertion unit through the slot and into the second lumen; and
a connector on said insertion unit adapted to selectively engage the flange of the base unit.

2. The endoscopic instrument management system of claim 1, further comprising a flexible endoscopic instrument extending through at least one of the plurality of flexible tubes.

3. The endoscopic instrument management system of claim 1, further comprising a pair of telescoping tubes disposed at a proximal end of at least one of the flexible tubes.

4. The endoscopic instrument management system of claim 3, wherein the pair of telescoping tubes comprise a first substantially rigid tube slidably received within a second substantially rigid tube, with one of the first substantially rigid tube or the second substantially rigid tube being attached to the proximal end of the at least one flexible tube.

5. The endoscopic instrument management system of claim 4, further comprising a flexible endoscopic instrument extending through the pair of telescoping tubes and the at least one flexible tube.

6. The endoscopic instrument management system of claim 5, further comprising a first interlock attached to a shaft of the flexible endoscopic instrument and a second interlock disposed at a proximal end of the pair of telescoping tubes, with the first interlock being selectively connectable to the second interlock.

7. The endoscopic instrument management system of claim 6, wherein the second interlock includes a sensor that receives a communication from the first interlock.

8. The endoscopic instrument management system of claim 7, wherein the sensor is electrical.

9. The endoscopic instrument management system of claim 7, wherein the sensor is mechanical.

10. The endoscopic instrument management system of claim 1, further comprising a steering control for the elongated conduit, said steering control comprising:
a handle having a steering actuator;
a flexible tether extending from a proximal end of the handle and attached to the elongated conduit; and
at least one tensioning wire extending from the handle through flexible tether and through the elongated conduit to a distal region of the elongated conduit.
